SEQUENCE LISTING (14 sequences)

SEQ ID NO: 1 - RNA, 23 nt, synthetic construct
  source 1..23: mol_type = other RNA
ctccagtctttctagaagatggg

SEQ ID NO: 2 - DNA, 27 bp, synthetic construct
  source 1..27: mol_type = other DNA
tctacctctttcctggcaatgactaca

SEQ ID NO: 3 - DNA, 24 bp, synthetic construct
  source 1..24: mol_type = other DNA
ctttattagccagaagtcagatgc

SEQ ID NO: 4 - DNA, 23 bp, synthetic construct
  source 1..23: mol_type = other DNA
cacttgctctcccaaagtcgctc

SEQ ID NO: 5 - DNA, 21 bp, synthetic construct
  source 1..21: mol_type = other DNA
atactccgaggcggatcacaa

SEQ ID NO: 6 - DNA, 21 bp, synthetic construct
  source 1..21: mol_type = other DNA
ccttcctgaagcagtagagca

SEQ ID NO: 7 - DNA, 20 bp, synthetic construct
  source 1..20: mol_type = other DNA
gccttattgtggaaggactg

SEQ ID NO: 8 - DNA, 21 bp, synthetic construct
  source 1..21: mol_type = other DNA
ttgctaaagcgctacatagga

SEQ ID NO: 9 - DNA, 24 bp, synthetic construct
  source 1..24: mol_type = other DNA
gattacaaggacgacgatgacaag

SEQ ID NO: 10 - protein, 8 aa, synthetic construct
DYKDDDDK

SEQ ID NO: 11 - DNA, 24 bp, synthetic construct
  source 1..24: mol_type = other DNA
tacgccacagggagtccaagaatg

SEQ ID NO: 12 - DNA, 25 bp, synthetic construct
  source 1..25: mol_type = other DNA
agatgtactgccaagtaggaaagtc

SEQ ID NO: 13 - DNA, 24 bp, synthetic construct
  source 1..24: mol_type = other DNA
gcatctgacttctggctaataaag

SEQ ID NO: 14 - DNA, 23 bp, synthetic construct
  source 1..23: mol_type = other DNA
ctggaaatcaggctgcaaatctc

(19) United States
(12) Patent Application Publication
(10) Pub. No.: US 2026/0072015 A1
(43) Pub. Date: Mar. 12, 2026

(54) Model for simulating ALS constructed based on CASP4 and its construction method

(71) Applicant: Jinan University, Guangzhou (CN)

(72) Inventors: Peng YIN, Guangzhou (CN); Xiaojiang LI, Guangzhou (CN); Qingqing JIA, Guangzhou (CN)

(73) Assignee: Jinan University, Guangzhou (CN)

(21) Appl. No.: 19/238,616

(22) Filed: Jun. 16, 2025

(30) Foreign Application Priority Data
Jun. 17, 2024 (CN) 2024107748074

(51) Int. Cl. G01N 33/50; A01K 67/0278; C12N 9/22; C12N 9/64; C12N 15/11; C12N 15/877; C12N 15/90

(52) U.S. Cl. CPC G01N 33/5091; A01K 67/0278; C12N 9/226; C12N 9/6472; C12N 15/11; C12N 15/8775; C12N 15/907; C12Y 304/22057; A01K 2217/052; A01K 2217/072; A01K 2227/105; A01K 2267/0356; C12N 2310/20

(57) ABSTRACT

An amyotrophic lateral sclerosis (ALS)-simulating model and a method for constructing the ALS-simulating model based on a caspase-4 (CASP4) gene are provideds. The method includes: (1) constructing a targeting fragment for knock-in of the CASP4 gene; (2) injecting gRNA, Cas9 mRNA, and the targeting fragment into a mouse zygote, culturing, and passaging to produce a hCASP4flox mouse with the CASP4 gene stably inherited; and (3) crossing the hCASP4flox mouse with a Cre driver mouse to produce a double-positive heterozygous mouse, which is a mouse model in which the CASP4 gene is specifically expressed in a nervous system. An ALS-simulating animal model is constructed based on a humanized CASP4 gene. The method can effectively avoid the mouse death caused by this apoptotic factor, and leads to an ALS-simulating mouse model in which TDP-43 fragments accumulate in the cytoplasm and TDP-43 is deleted in the nucleus.

CROSS REFERENCE TO THE RELATED APPLICATIONS

This application is based upon and claims priority to Chinese Patent Application No. 202410774807.4, filed on Jun. 17, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBCD180-PKGG_SequenceListing.xml, created on Jul. 7, 2025, and is 13,515 bytes in size.

### TECHNICAL FIELD

The present disclosure belongs to the field of biotechnologies, and specifically relates to an amyotrophic lateral sclerosis (ALS)-simulating model and a method for constructing the ALS-simulating model based on a caspase-4 (CASP4) gene.

### BACKGROUND

With the demand for precision medicine, providing reliable humanized animal models for disease research and new drug screening has become both a hot spot and a challenge in the field of experimental animal models in recent years. Transactive response DNA-binding protein 43 (TDP-43, TARDBP) is a multifunctional DNA- and RNA-binding protein that plays a crucial role in processes such as RNA transcription, alternative splicing, and regulation of mRNA stability in nuclei. In the brains of ALS patients, TDP-43 abnormally relocates from the neuronal nucleus to the cytoplasm and aggregates, which serves as both a key marker and a pathogenic mechanism for ALS. There is the loss of normal functions of endogenous TDP-43 in the nucleus (loss of function). The aberrant interaction of TDP-43 with other functional proteins in the cytoplasm further leads to acquired neurotoxicity (gain of function). When the “loss-of-function” and the “gain-of-function” accumulate to a critical threshold, ALS patients experience degeneration and death of upper and lower motor neurons in the central nervous system, resulting in muscle atrophy and ultimately the gradual loss of the brain's movement-control ability.

Therefore, the establishment of an experimental mouse model in which the pathological relocation of TDP-43 from the nucleus to the cytoplasm in the brain is simulated is crucial for the treatment and research of ALS. So far, there has been no mouse model that can simultaneously demonstrate the disease features of the loss of TDP-43 in the nucleus and the aggregation of TDP-43 in the cytoplasm. In all of the current TDP-43-transgenic rodent models, TDP-43 accumulates exclusively in the neuronal nucleus in a full-length form (Shan, X. (2010). Altered distributions of Gemini of coiled bodies and mitochondria in motor neurons of TDP-43 transgenic mice. Proceedings of the National Academy of Sciences.) (Mitchell, J.C. (2015). Wild type human TDP-43 potentiates ALS-linked mutant TDP-43 driven progressive motor and cortical neuron degeneration with pathological features of ALS. Acta neuropathologica communications.). Alternatively, in some TDP-43 fragment-transgenic mice, although TDP-43 fragments can accumulate in the cytoplasm, the critical pathological feature of nuclear TDP-43 loss still cannot be demonstrated simultaneously (Antonella Caccamo. (2012) Cognitive decline typical of frontotemporal lobar degeneration in transgenic mice expressing the 25-kDa C-terminal fragment of TDP-43. Am J Pathol.) (Antonella Caccamo. (2015) Reduced protein turnover mediates functional deficits in transgenic mice expressing the 25 kDa C-terminal fragment of TDP-43. Hum Mol Genet.). As a result, the data acquired from these mouse models and the drugs developed accordingly can hardly be used for ALS patients.

Caspase-4 (CASP4) is a hydrolase expressed only in higher animals such as humans and monkeys. Under pathological conditions, the CASP4-mediated hydrolysis can achieve the cleavage of various substrate proteins, including pro-interleukin 18 (pro-IL-18). In contrast, the mouse homolog caspase-11 (CASP11) cannot achieve this cleavage effect (Xuyan Shi. (2023). Recognition and maturation of IL-18 by caspase-4 noncanonical inflammasome. Nature.) (Pascal Devant. (2023). Structural insights into cytokine cleavage by inflammatory caspase-4. Nature.).

If a mouse model with the conditional knock-in of the CASP4 gene can be established, the translocation of the endogenous TDP-43 protein from the nucleus to the cytoplasm in mice can be accurately achieved, and ALS can be well investigated with mouse resources. Accordingly, the excessive gene copy number drawback caused by the overexpression of TDP-43 or fragments thereof can be avoided. However, there has not been any CASP4 knock-in mouse model. Because CASP4 is a member of the inflammatory caspase subfamily. Various studies have shown that the transient expression of CASP4 will induce the apoptosis of various cell lines (Hitomi, J, (2004). Involvement of caspase-4 in endoplasmic reticulum stress-induced apoptosis and Abeta-induced cell death. J. Cell Biol.) (Mao, Z.G., (2010). TRAIL-induced apoptosis of human melanoma cells involves activation of caspase-4. Apoptosis.). Therefore, how to construct an ALS-simulating animal model based on the CASP4 gene is particularly crucial in the disease research and drug development for ALS.

### SUMMARY

In view of the above deficiencies in the prior art, the present disclosure provides an ALS-simulating model and a method for constructing the ALS-simulating model based on a CASP4 gene. The present disclosure can effectively simulate the progression of ALS and avoid the cell apoptosis caused by the CASP4 gene.

To achieve the above objective, the present disclosure adopts the following technical solutions to solve the technical problems of the present disclosure:

A method for constructing an ALS-simulating model based on a CASP4 gene is provided, including the following steps:
(1) constructing a targeting fragment CAG-LSL-human CASP4 (hCASP4)-posttranscriptional regulatory element of woodchuck hepatitis virus (WPRE)-polyA for knock-in of the CASP4 gene;
(2) injecting gRNA, Cas9 mRNA, and the targeting fragment CAG-loxP-stop-loxP (LSL)-hCASP4-WPRE-polyA into a mouse zygote, culturing, and passaging to produce a hCASP4^{flox} mouse with the CASP4 gene stably inherited; and
(3) crossing the hCASP4^{flox}mouse with a Cre driver mouse to produce a double-positive heterozygous mouse, which is a mouse model in which the CASP4 gene is specifically expressed in a nervous system.

Further, a process for constructing the targeting fragment

CAG-LSL-hCASP4-WPRE-polyA is as follows:
inserting a loxP-PGK-Neo-6*SV40pA-loxP expression cassette (LSL), hCASP4, WPRE, and a polyA sequence into a plasmid carrying a CAG promoter to produce the targeting fragment.

Further, a process for constructing the loxP-PGK-Neo-6*SV40pA-loxP expression cassette is as follows:

The expression cassette includes two loxP sites between which there is a PGK promoter-driven neomycin resistance gene (Neo) and six SV40 polyadenylation signal sequences (SV40pA). This expression cassette is provided to enable the expression of the Neo gene through Cre recombinase, thereby inducing the activation or silencing of the inserted hCASP4 gene.

Through the Cre recombinase-mediated loxP site recombination, the inserted gene can be selectively activated or silenced to achieve the manipulation of the hCASP4 gene.

Further, a sequence of the gRNA is CTCCAGTCTTTCTAGAAGAT-GGG (SEQ ID NO: 1). Further, the CASP4 gene is a humanized CASP4 gene CASP4-201 with a sequence identifier of ENST00000444739.7.

Further, the mouse zygote is derived from a C57BL/6JGp mouse.

Further, a process for acquiring the hCASP4^{flox} mouse with the CASP4 gene stably inherited is as follows:
transplanting a viable zygote undergoing the injection into a pseudopregnant female mouse, and culturing to produce F0 mice; identifying through sequencing to produce F0 positive hCASP4^{flox} mice; and crossing the F0 positive hCASP4^{flox} mice to produce a F1 hCASP4^{flox} mouse model with the CASP4 gene stably inherited.

Further, amplification primers for acquiring the F0 positive hCASP4^{flox} mice are as follows:

Caspase-4-F-B1:
(SEQ ID NO: 11)
5′-TACGCCACAGGGAGTCCAAGAATG-3′;
Caspase-4-R-B1:
(SEQ ID NO: 12)
5′-AGATGTACTGCCAAGTAGGAAAGTC-3′;
Caspase-4-F-B2:
(SEQ ID NO: 13)
5′-GCATCTGACTTCTGGCTAATAAAG-3′;
and
Caspase-4-R-B2:
(SEQ ID NO: 14)
5′-CTGGAAATCAGGCTGCAAATCTC-3′;

and
a polymerase chain reaction (PCR) program is as follows: pre-denaturation at 94° C. for 3 min, denaturation at 94° C. for 30 s, annealing at 60° C. for 30 s, and extension at 65° C. for 50 s per kb, with 33 cycles; and extension at 65° C. for 10 min. A PCR product is stored at 4° C. Further, the Cre driver mouse is a Nestin-Cre mouse.

Further, amplification primers for identifying the double-positive heterozygous mouse are as follows:

Caspase-4-F-C1:
(SEQ ID NO: 2)
5′-TCTACCTCTTTCCTGGCAATGACTACA-3′;
Caspase-4-R-C1:
(SEQ ID NO: 3)
5′-CTTTATTAGCCAGAAGTCAGATGC-3′;
Caspase-4-F-C2:
(SEQ ID NO: 4)
5′-CACTTGCTCTCCCAAAGTCGCTC-3′;
Caspase-4-R-C2:
(SEQ ID NO: 5)
5′-ATACTCCGAGGCGGATCACAA-3′;
Nestin-F-N1:
(SEQ ID NO: 6)
5′-CCTTCCTGAAGCAGTAGAGCA-3′;
Nestin-R-N:
(SEQ ID NO: 7)
5′-GCCTTATTGTGGAAGGACTG-3′;
and
Nestin-F-N2:
(SEQ ID NO: 8)
5′-TTGCTAAAGCGCTACATAGGA-3′.

An ALS-simulating mouse model constructed by the method described above is provided.

A use of the ALS-simulating mouse model described above as an animal model in screening drugs for preventing and treating ALS and/or in investigating clinical occurrence and development of ALS is provided.

The present disclosure has the following beneficial effects:

In the present disclosure, the humanized CASP4 gene is silenced in FO mice through the loxP-PGK-Neo-6*SV40pA-loxP expression cassette, and then silenced mice are crossed with Cre driver mice, so as to achieve the targeted expression of the humanized CASP4 gene in the mouse nervous system. The present disclosure can effectively avoid the mouse death caused by this apoptotic factor, and leads to an ALS-simulating mouse model in which TDP-43 fragments accumulate in the cytoplasm and TDP-43 is deleted in the nucleus.

### BRIEF DESCRIPTION OF THE DRAWINGS

FIG. 1 is a map of a constructed targeting fragment CAG-LSL-hCASP4-WPRE-polyA;

FIG. 2 shows the electrophoresis results for testing of knock-in of a target gene in F1 positive mice;

FIG. 3 shows the gel electrophoresis results of PCR products of genomic DNAs from double-positive heterozygous mice;

FIG. 4 shows the immunofluorescence staining results of the ALS-simulating mouse model constructed in the present disclosure;

FIG. 5 shows the immunohistochemical staining results of the ALS-simulating mouse model constructed in the present disclosure;

FIG. 6 shows the motor behavioral testing results of the ALS-simulating mouse model constructed in the present disclosure;

FIG. 7 shows the hindlimb muscle morphology testing results of the ALS-simulating mouse model constructed in the present disclosure;

FIGS. 8A-8B show the gene expression profile analysis results of the ALS-simulating mouse model constructed in the present disclosure; and

FIG. 9 shows the immunofluorescence staining results of markers in the ALS-simulating mouse model constructed in the present disclosure.

### DETAILED DESCRIPTION OF THE EMBODIMENTS

The specific embodiments of the present disclosure will be described below to make those skilled in the art easily understand the present disclosure, but it should be noted that the present disclosure is not limited to the scope of the specific embodiment. For those of ordinary skill in the art, as long as various changes fall within the spirit and scope of the present disclosure defined and determined by the appended claims, these changes are apparent, and all inventions and creations using the concept of the present disclosure are protected.

### Example 1 Construction of a Targeting Fragment CAG-LSL-hCASP4-WPRE-polyA

### 1. Information of a Knock-In Gene

The knock-in gene was a humanized CASP4 gene CASP4-201 with a sequence identifier of ENST00000444739.7.

### 2. Construction of a CAG-LSL-hCASP4-WPRE-polyA Fragment

(1) Construction of a loxP-PGK-Neo-6*SV40pA-loxP expression cassette (LSL)

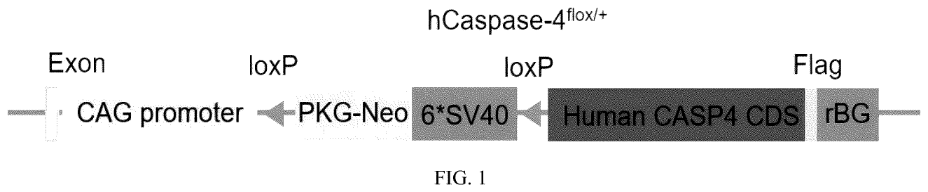

The expression cassette included two loxP sites between which there was a PGK promoter-driven neomycin resistance gene (Neo) and six SV40 polyadenylation signal sequences (SV40pA).
(2) A plasmid that included a CAG strong promoter and could stably replicate in cells was selected, and then the loxP-PGK-Neo-6*SV40pA-loxP expression cassette, hCASP4, WPRE, and a polyA sequence were inserted into the plasmid.
(3) A protein tag 3XFLAG sequence was ligated to the humanized gene CASP4. The FLAG tag was a polypeptide composed of 8 amino acids: N-DYKDDDDK-C (1,012 Da) (SEQ ID NO: 10), and a gene sequence encoding the FLAG tag was as follows: GATTACAAGGACGACGATGACAAG (SEQ ID NO: 9). A vector finally constructed was shown in FIG. 1.

### Example 2 Construction of an Animal Model (hCASP4 Mice)

### 1. Breeding of F1 Positive Individuals

gRNA, Cas9 mRNA, and CAG-LSL-hCASP4-WPRE-polyA were injected into C57BL/6JGp mouse zygotes. Viable zygotes undergoing the injection were collected and transplanted into pseudopregnant female mice. The humanized CASP4 gene was knocked into the Rosa26 locus on chromosome 6 of the mice. Mice undergoing transplantation were cultured to produce F0 positive hCASP4^{flox} mice. The FO mice were further crossed to produce F1positive individuals hCasp4^{flox} with the CASP4 gene stably inherited. Primers for screening and identification were as follows:

Caspase-4-F-B1:
(SEQ ID NO: 11)
5′-TACGCCACAGGGAGTCCAAGAATG-3′;
Caspase-4-R-B1:
(SEQ ID NO: 12)
5′-AGATGTACTGCCAAGTAGGAAAGTC-3′;
Caspase-4-F-B2:
(SEQ ID NO: 13)
5′-GCATCTGACTTCTGGCTAATAAAG-3′;
and
Caspase-4-R-B2:
(SEQ ID NO: 14)
5′-CTGGAAATCAGGCTGCAAATCTC-3′.

A PCR program was as follows: pre-denaturation at 94° C. for 3 min, denaturation at 94° C. for 30 s, annealing at 60° C. for 30 s, and extension at 65° C. for 50 s per kb, with 33 cycles; and extension at 65° C. for 10 min. A PCR product was stored at 4° C. A PCR system was shown in Table 1.

TABLE 1
PCR system for identifying F1 positive
mouse individuals hCasp4^{flox}

| Component | Amount (μL) |
|---|---|
| Mouse tail genomic DNA | 2 |
| Forward primer (10 μM) | 2 |
| Reverse primer (10 μM) | 2 |
| dNTPs (2.5 mM) | 6 |
| 5 × LongAmp Taq Reaction | 10 |
| LongAmp Taq DNA Polymerase | 2 |
| ddH₂O | 26 |
| Total | 50 |

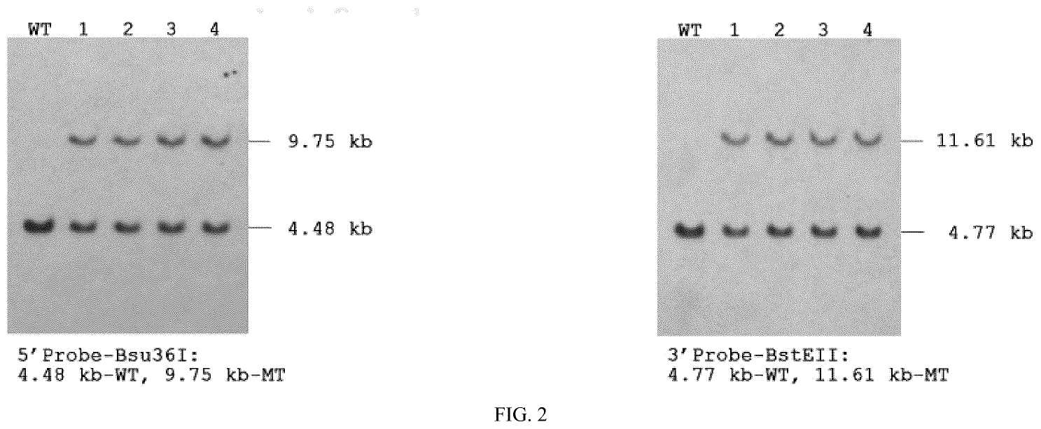

A tail DNA sample was subjected to Southern blot analysis with 5′ and 3′ probes to verify the correct gene targeting in the F1 positive mice. Results were shown in FIG. 2.

As shown in FIG. 2, the target gene had been correctly inserted in four mice (1, 2, 3, and 4). The next model construction was then conducted.

### 2. Breeding of Double-Positive Heterozygous Mice

The loxP-PGK-Neo-6*SV40pA-loxP expression cassette was integrated into the F1 positive hCASP4^{flox} mice. Mice with the expression cassette integrated were then crossed with Nestin-Cre driver mice. Screening was conducted to obtain double-positive heterozygous mice, which was a Nestin-Cre⁺ and hCaspase-4^{flox/+} mouse model in which the humanized CASP4 gene underwent targeted expression in the mouse nervous system. Specific amplification primers for screening and identification were as follows:

Primers for identifying the genomic DNA of Caspase-4^{flox/+} mice were as follows:

Caspase-4-F-C1:
(SEQ ID NO: 2)
5′-TCTACCTCTTTCCTGGCAATGACTACA-3′;
Caspase-4-R-C1:
(SEQ ID NO: 3)
5′-CTTTATTAGCCAGAAGTCAGATGC-3′;
Caspase-4-F-C2:
(SEQ ID NO: 4)
5′-CACTTGCTCTCCCAAAGTCGCTC-3′;
and
Caspase-4-R-C2:
(SEQ ID NO: 5)
5′-ATACTCCGAGGCGGATCACAA-3′.

A PCR program was as follows: pre-denaturation at 94° C. for 3 min, denaturation at 94° C. for 30 s, annealing at 60° C. for 35 s, and extension at 72° C. for 35 s, with 35 cycles; and extension at 72° C. for 5 min. A PCR product was stored at 4° C. A PCR system was shown in Table 2.

TABLE 2
PCR system for identifying Caspase-4^{flox/+} mice

| Component | Amount (μL) |
|---|---|
| 10 × PCR buffer | 2.5 |
| 2.5 mM dNTPs | 2 |
| TaKaRa rTaq | 0.25 |
| 10 μM Primer forward (F-C1) | 1 |
| 10 μM Primer reverse (R-C1) | 1 |
| 10 μM Primer forward (F-C2) | 1 |
| 10 μM Primer reverse (R-C2) | 1 |
| H₂O | 14.75 |
| Template DNA | 1.5 |
| Total | 25 |

Primers for identifying the genomic DNA of Nestin-Cre⁺ mice were as follows:

Nestin-F-N1:
(SEQ ID NO: 6)
5′-CCTTCCTGAAGCAGTAGAGCA-3′;
Nestin-R-N1:
(SEQ ID NO: 7)
5′-GCCTTATTGTGGAAGGACTG-3′;
and
Nestin-F-N2:
(SEQ ID NO: 8)
5′-TTGCTAAAGCGCTACATAGGA-3′.

A PCR program was as follows: pre-denaturation at 94° C. for 4 min, denaturation at 94° C. for 30 s, annealing at 60° C. for 45 s, and extension at 72° C. for 1 min, with 32 cycles; and extension at 72° C. for 10 min. A PCR product was stored at 4° C. A PCR system was shown in Table 3.

TABLE 3
PCR system for identifying Nestin-Cre mice

| Component | Amount (μL) |
|---|---|
| 10 × PCR buffer | 2 |
| 2.5 mM dNTPs | 1.6 |
| TaKaRa rTaq | 0.25 |
| 10 μM Primer forward (F-N1) | 1 |
| 10 μM Primer reverse (R-N) | 1 |
| 10 μM Primer forward (F-N2) | 1 |
| H₂O | 10.15 |
| Template DNA | 3 |
| Total | 20 |

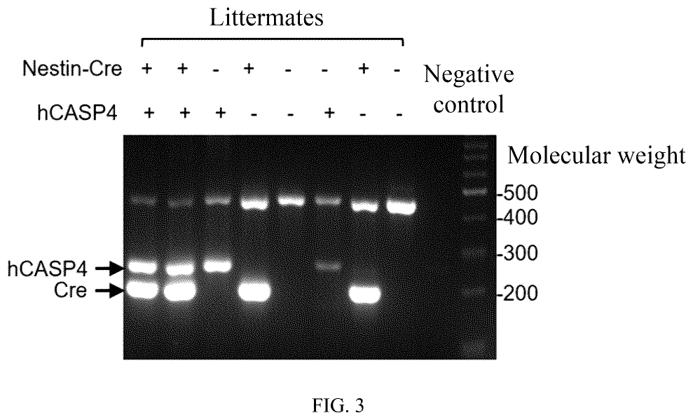

3. The double-positive heterozygous mice were identified, and results were shown in FIG. 3. As shown in FIG. 3, a proportion of the humanized gene hCASP4/Cre gene double-positive mice among littermates was 25%, which was consistent with the Mendel's law of inheritance.

### Example 3

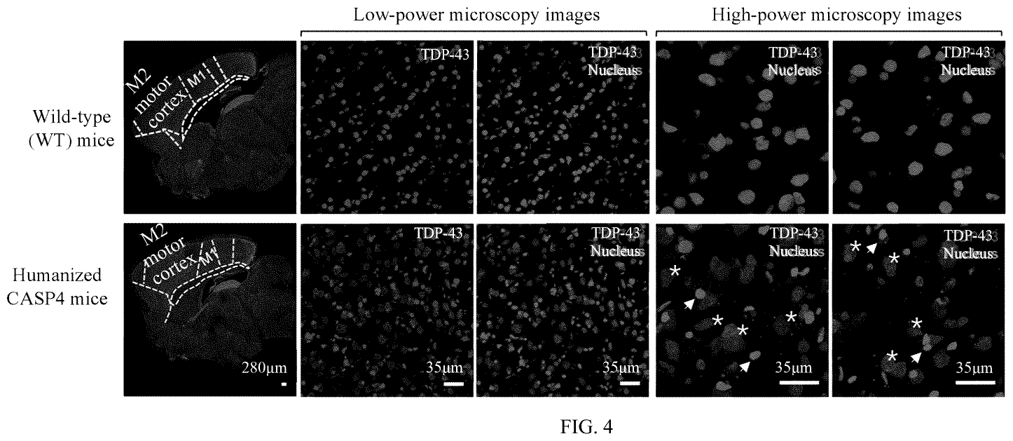

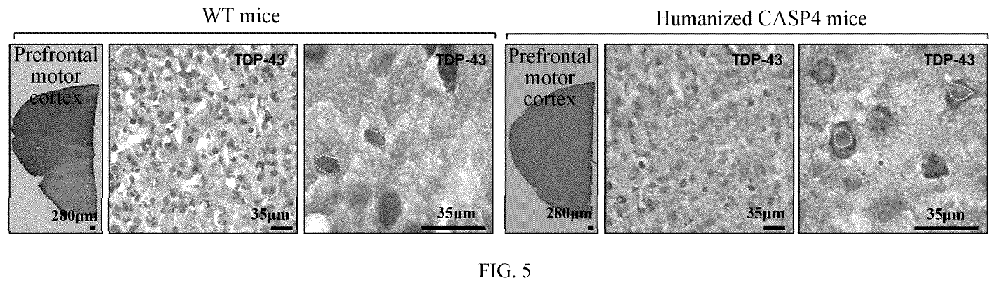

In the present disclosure, a protein tag 3XFLAG sequence was ligated to the humanized gene CASP4. The FLAG tag was a polypeptide composed of 8 amino acids: N-DYKDDDDK-C (1,012 Da) (SEQ ID NO: 10), and a gene sequence encoding the FLAG tag was as follows: GATTACAAGGACGACGATGACAAG (SEQ ID NO: 9). Therefore, the animal model constructed in Example 2 and wild-type (WT) mice each were subjected to fluorescence staining, and test results were shown in FIG. 4 and FIG. 5.

As shown in FIG. 4 and FIG. 5, TDP-43 was expressed in the nucleus in WT mice, while endogenous TDP-43 relocated from the nucleus to the cytoplasm in the animal model constructed in the present disclosure, further indicating that the animal model desired by the present disclosure was successfully established.

### Example 4

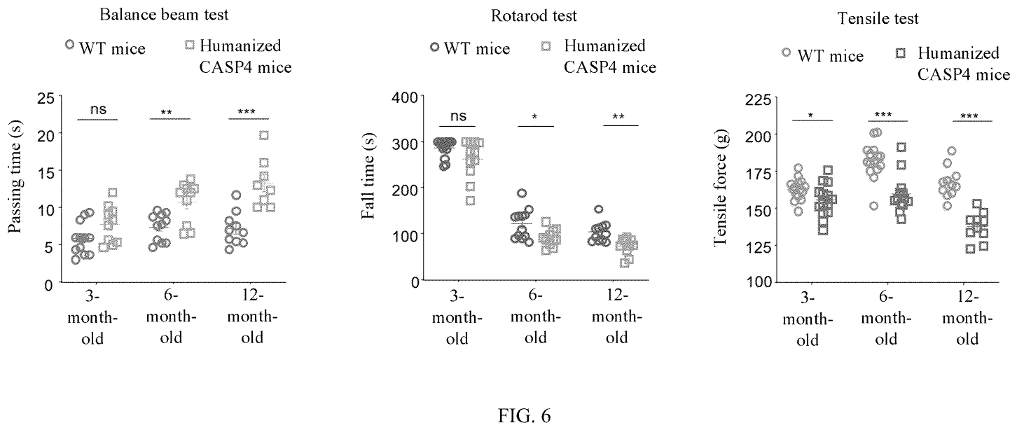

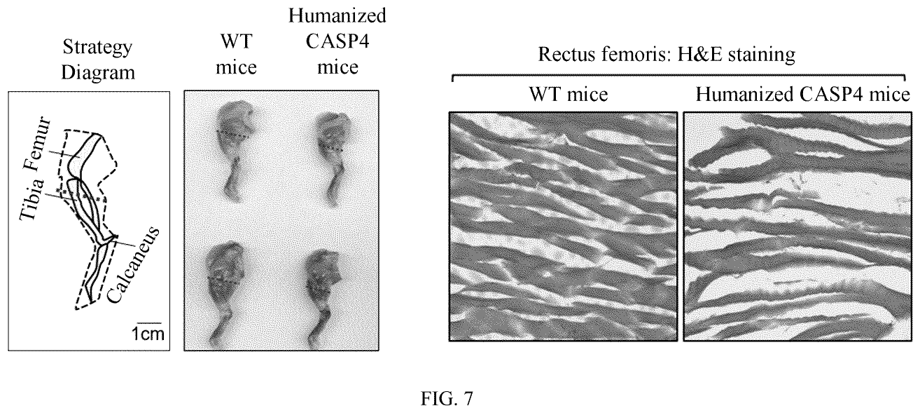

1. The animal model constructed in Example 2 of the present disclosure was subjected to motor behavioral tests, including rotarod, tensile, and balance beam tests, and a muscle morphology test. With WT mice as a control, it was determined whether the animal model could simulate the motor dysfunction in ALS patients. Results were shown in FIG. 6 and FIG. 7.

As shown in FIG. 6 and FIG. 7, compared with the WT mice, the animal model constructed in the present disclosure successfully simulated the motor dysfunction in ALS patients, and underwent the consistent muscle atrophy symptoms in lower limbs with ALS patients.

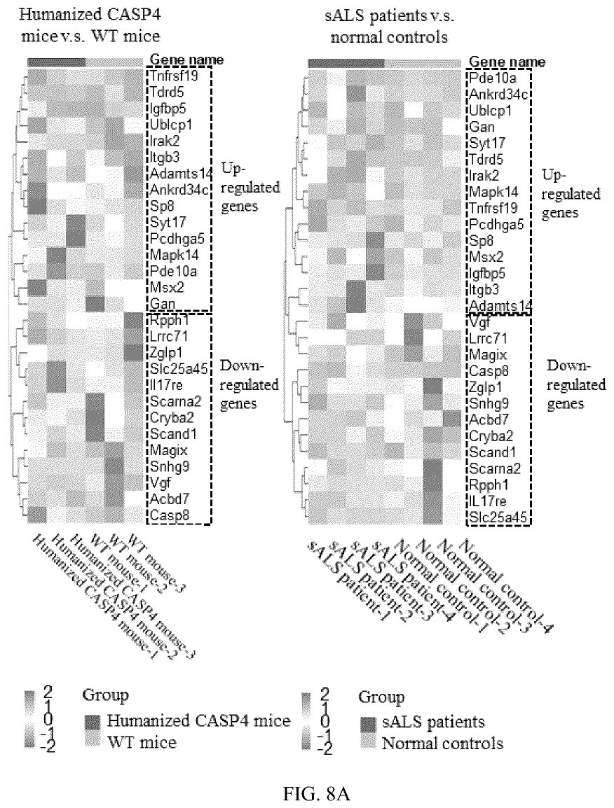

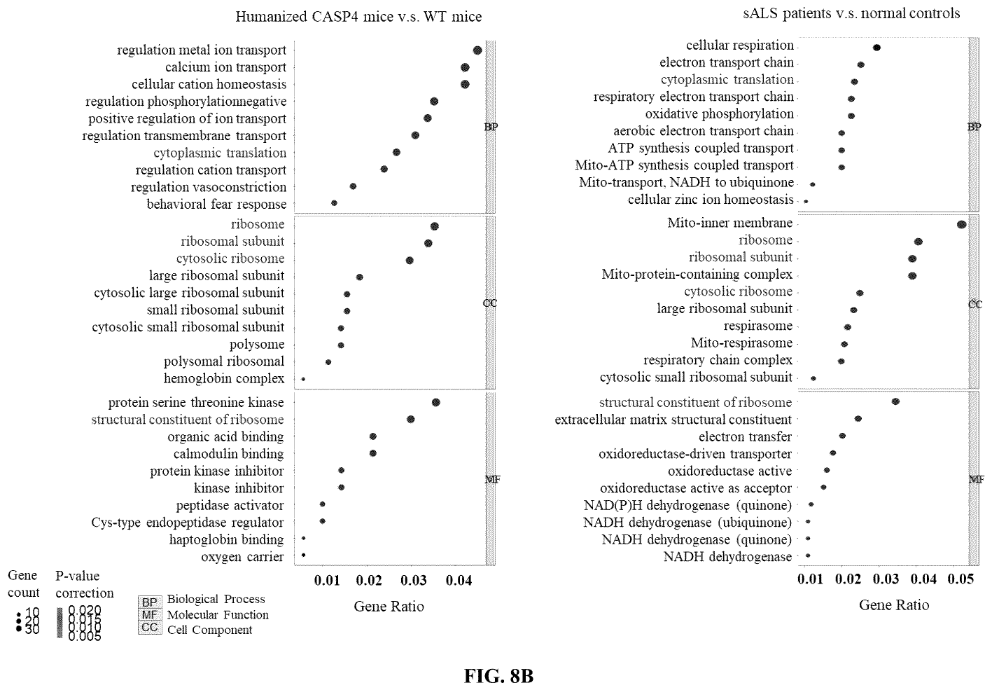

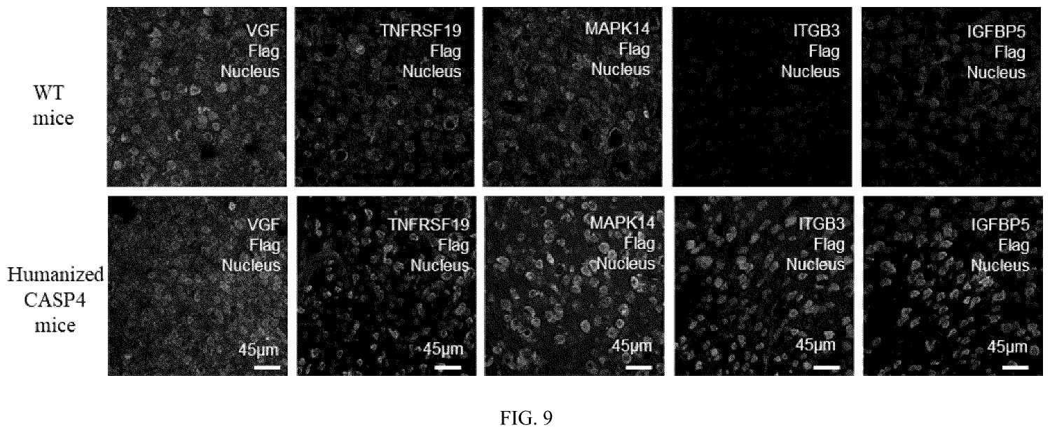

2. The animal model (hCASP4 mice) constructed in Example 2 of the present disclosure was subjected to gene expression profile analysis and marker detection. Results were shown in FIGS. 8A-8B and FIG. 9.

As shown in FIGS. 8A-8B, a gene expression difference of the overall transcript in the prefrontal cortex between the WT mice and the hCASP4 mice was subjected to volcano plot analysis, and then compared with the overall transcript gene change in sALS patients for similarity. The publicly-available mRNA-seq data (GSE67196) was used for the comparison between RNA from the prefrontal cortex of sALS patients without mutations in the most common ALS-associated genes and RNA from healthy individuals without nervous system diseases. It was found that there was a specified similarity in gene expression between the mouse model and ALS patients.

Moreover, differentially expressed genes in prefrontal cortices of healthy individuals and sALS patients were subjected to gene ontology (GO) analysis. It was found that the differentially expressed genes in the prefrontal cortices of the healthy individuals and sALS patients were clustered in the biological process (BP) pathway of “cytoplasmic translation”, the cellular component (CC) pathways of “ribosome”, “ribosomal subunit”, and “cytoplasmic ribosome”, and the molecular function (MF) pathway of “structural constituent of ribosome.” These differentially expressed genes underwent similar clustering in the mouse model.

As shown in FIG. 9, in the mouse model, the expression levels of VGF, ITGB3, Mapk14, IGFBP5, and TNFRSF19 proteins as markers for detecting ALS changed in the same trend as reported in ALS patients.

In summary, an ALS-simulating mouse model in which TDP-43 fragments accumulate in the cytoplasm and TDP-43 is deleted in the nucleus is successfully established based on the CASP4 gene in the present disclosure. The ALS-simulating mouse model is expected to become a prominent experimental animal model for investigating molecular mechanisms and therapeutic strategies for TDP-43-associated diseases.

It should be noted that the above embodiments are only intended to explain, rather than to limit the technical solutions of the present disclosure. Although the present disclosure is described in detail with reference to the embodiments, those of ordinary skill in the art should understand that modifications or equivalent substitutions may be made to the technical solutions of the present disclosure without departing from the spirit and scope of the technical solutions of the present disclosure, and such modifications or equivalent substitutions should be included within the scope of the claims of the present disclosure. What is claimed is:

1-10. (canceled)

11. A method for constructing an amyotrophic lateral sclerosis (ALS)-simulating model based on a caspase-4 (CASP4) gene, comprising the following steps:
(1) constructing a targeting fragment CAG-loxP-stop-loxP (LSL)-human CASP4(hCASP4)-posttranscriptional regulatory element of woodchuck hepatitis virus (WPRE)-polyA for a knock-in of the CASP4 gene;
(2) injecting a gRNA, a Cas9 mRNA, and the targeting fragment CAG-LSL-hCASP4-WPRE-polyA into a mouse zygote, culturing, and passaging to produce a hCASP4^{flox} mouse with the CASP4 gene stably inherited; and
(3) crossing the hCASP4^{flox} mouse with a Nestin-Cre driver mouse to produce a double-positive heterozygous mouse, namely, a mouse model, wherein in the mouse model, the CASP4 gene is specifically expressed in a nervous system;
wherein amplification primers for identifying the double-positive heterozygous mouse are as follows:
Caspase-4-F-C1:
5′-TCTACCTCTTTCCTGGCAATGACTACA-3′,
as shown in SEQ ID NO: 2;
Caspase-4-R-C1:
5′-CTTTATTAGCCAGAAGTCAGATGC-3′,
as shown in SEQ ID NO: 3;
Caspase-4-F-C2:
5′-CACTTGCTCTCCCAAAGTCGCTC-3′,
as shown in SEQ ID NO: 4;
Caspase-4-R-C2:
5′-ATACTCCGAGGCGGATCACAA-3′,
as shown in SEQ ID NO: 5;
Nestin-F-N1:
5′-CCTTCCTGAAGCAGTAGAGCA-3′,
as shown in SEQ ID NO: 6;
Nestin-R-N:
5′-GCCTTATTGTGGAAGGACTG-3′,
as shown in SEQ ID NO: 7; and
Nestin-F-N2:
5′-TTGCTAAAGCGCTACATAGGA-3′,
as shown in SEQ ID NO: 8.

12. The method for constructing the ALS-simulating model based on the CASP4 gene according to claim 11, wherein a process for constructing the targeting fragment CAG-LSL-hCASP4-WPRE-polyA is as follows:
ligating a CAG promoter, a loxP-PGK-Neo-6*SV40pA-loxP expression cassette, hCASP4, a WPRE, and a polyA sequence to produce the targeting fragment CAG-LSL-hCASP4-WPRE-polyA.

13. The method for constructing the ALS-simulating model based on the CASP4 gene according to claim 11, wherein the is sequence of the gRNA CTCCAGTCTTTCTAGAAGAT-GGG, as shown in SEQ ID NO:1.

14. The method for constructing the ALS-simulating model based on the CASP4 gene according to claim 11, wherein the CASP4 gene has a sequence identifier of ENST00000444739.7.

15. The method for constructing the ALS-simulating model based on the CASP4 gene according to claim 11, wherein a process for acquiring the hCASP4flox mouse with the CASP4gene stably inherited is as follows:
transplanting a viable zygote undergoing an injection into a pseudopregnant female mouse, and culturing to produce F0 mice; identifying through sequencing to produce F0 positive hCASP4^{flox} mice; and crossing the F0 positive hCASP4^{flox} mice to produce a F1 hCASP4^{flox} mouse model with the CASP4 gene stably inherited.

16. The method for constructing the ALS-simulating model based on the CASP4 gene according to claim 15, wherein amplification primers for acquiring the F0 positive hCASP4^{flox} mice are as follows:
Caspase-4-F-B1:
5′-TACGCCACAGGGAGTCCAAGAATG-3′,
as shown in SEQ ID NO: 11;
Caspase-4-R-B1:
5′-AGATGTACTGCCAAGTAGGAAAGTC-3′,
as shown in SEQ ID NO: 12;
Caspase-4-F-B2:
5′-GCATCTGACTTCTGGCTAATAAAG-3′,
as shown in SEQ ID NO: 13; and
Caspase-4-R-B2:
5′-CTGGAAATCAGGCTGCAAATCTC-3′,
as shown in SEQ ID NO: 14; and
a polymerase chain reaction (PCR) program is as follows: a pre-denaturation at 94° C. for 3min, a denaturation at 94° C. for 30 s, annealing at 60° C. for 30 s, and a first extension at 65° C. for 50 s per kb, with 33 cycles; and a second extension at 65° C. for 10 min.

17. A use of a mouse model constructed by the method according to claim 11 in constructing an ALS model simulating an intranuclear deletion of a transactive response DNA-binding protein 43 (TDP-43).

18. The use according to claim 17, wherein in the method, a process for constructing the targeting fragment CAG-LSL-hCASP4-WPRE-polyA is as follows:
ligating a CAG promoter, a loxP-PGK-Neo-6*SV40pA-loxP expression cassette, hCASP4, a WPRE, and a polyA sequence to produce the targeting fragment CAG-LSL-hCASP4-WPRE-polyA.

19. The use according to claim 17, wherein in the method, the sequence of the gRNA is CTCCAGTCTTTCTAGAAGAT-GGG, as shown in SEQ ID NO: 1.

20. The use according to claim 17, wherein in the method, the CASP4 gene has a sequence identifier of ENST00000444739.7.

21. The use according to claim 17, wherein in the method, a process for acquiring the hCASP4^{flox} mouse with the CASP4 gene stably inherited is as follows:
transplanting a viable zygote undergoing the injecting into a pseudopregnant female mouse, and culturing to produce F0 mice; identifying through sequencing to produce F0 positive hCASP4^{flox} mice; and crossing the F0 positive hCASP4^{flox} mice to produce a F1 hCASP4^{flox} mouse model with the CASP4 gene stably inherited.

22. The use according to claim 21, wherein in the method, amplification primers for acquiring the F0 positive hCASP4^{flox} mice are as follows:
Caspase-4-F-B1:
5′-TACGCCACAGGGAGTCCAAGAATG-3′,
as shown in SEQ ID NO: 11;
Caspase-4-R-B1:
5′-AGATGTACTGCCAAGTAGGAAAGTC-3′,
as shown in SEQ ID NO: 12;
Caspase-4-F-B2:
5′-GCATCTGACTTCTGGCTAATAAAG-3′,
as shown in SEQ ID NO: 13; and
Caspase-4-R-B2:
5′-CTGGAAATCAGGCTGCAAATCTC-3′,
as shown in SEQ ID NO: 14;
and
a PCR program is as follows: a pre-denaturation at 94° C. for 3 min, a denaturation at 94° C. for 30 s, annealing at 60° C. for 30 s, and a first extension at 65° C. for 50 s per kb, with 33 cycles;
and a second extension at 65° C. for 10 min.
